(19) United States
(12) Plant Patent Application Publication
(10) Pub. No.: US 2003/0014787 P1
(43) Pub. Date: Jan. 16, 2003

(54) Floribunda rose variety 'POULfeld'

(76) Inventors: L. Pernille Olesen, Fredensborg (DK); Mogens N. Olesen, Fredensborg (DK)

Correspondence Address: Poulsen Roser Pacific, Inc., 620 S. Front Street, Central Point, OR, 97502, US

(21) Appl. No.: 10/155,849

(22) Filed: May 24, 2002

Related U.S. Application Data
(63) Continuation of application No. 09/287,293, filed on Mar. 31, 1999.

(51) Int. Cl. A01H 5/00

(52) U.S. Cl. PLT/144

(57) ABSTRACT

A new garden rose plant which has abundant, white flowers and attractive foliage. This new and distinct variety has shown to be uniform and stable in the resulting generations from asexual propagation.

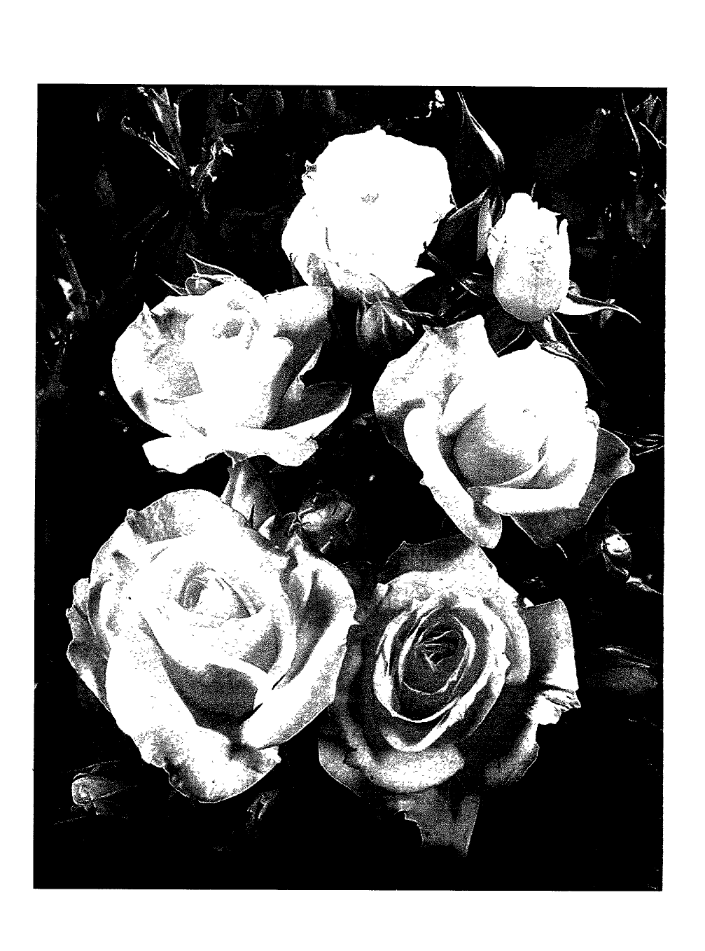

### SUMMARY OF THE INVENTION

The present invention constitutes a new and distinct variety of garden rose plant which originated from a controlled crossing made in summer 1985 between an unnamed seeding and an unnamed seedling. The two parents were crossed and the resulting seeds were planted in a controlled environment. The new variety is named ‘POULfeld’.

The new rose may be distinguished from its seed parent, an unnamed seedling hybridized by the same inventors, by the following combination of characteristics:

The seed parent has small white flowers, while ‘POULgeld’ has larger white flowers.

The new variety may be distinguished from its pollen parent, an unnamed seedling created by the same inventors, by the following combination of characteristics:

The pollen parent is a patio rose with creamy, white flowers which are smaller than the flowers of ‘POULfeld’.

The objective of the hybridization of this rose variety for garden use was to create a new and distinct variety with unique qualities, such as:

1. Uniform and abundant flowers;

2. Vigorous, yet compact growth;

3. Cold hardiness and disease resistance.

This combination of qualities is not present in previously available commercial cultivars of this type and distinguishes ‘POULfeld’ from all other varieties of which we are aware.

As part of their rose development program, L. Pemille Olesen and Mogens N. Olesen germinated the seeds from the aforementioned hybridization and conducted evaluations on the resulting seedlings in a controlled environment in Fredensborg, Denmark. ‘POULfeld’ was selected by the inventors in the spring of 1986 as a single plant from the progeny of the aforementioned hybridization.

Asexual reproduction of ‘POULfeld’ by traditional budding was first done by L. Pernille and Mogens N. Olesen in August, 1986. This initial and other subsequent propagations conducted in controlled environments have demonstrated that the characteristics of ‘POULfeld’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, and stems of ‘POULfeld’. Specifically illustrated in SHEET 1:

1. Stem showing branching and the attachment of leaves, buds, and peduncles;

2. Flower bud, partially opened bud, and open bloom;

3. Flower petals, detached;

Specifically illustrated in SHEET 2:

4. Sepals, receptacle, and pedicel;

5. Flowering stem as well as a bare stem exhibiting thorns;

6. Leaves.

### DETAILED DESCRIPTION OF THE VARIETY

The following is a description of ‘POULfeld’, as observed in its outdoor growth in a field nursery in Jackson County, Oregon. Observations were conducted during October, 1998. Color references are made using The Royal Horticultural Society (London, England) Colour Chart, 1995, except where common terms of color are used.

For a comparison, several physical characteristics of the rose variety ‘POULander’, a rose variety from the same inventors described and illustrated in U.S. Plant Pat. No. 6,265 and issued on Aug. 30, 1988 are compared to ‘POULfeld’ in Chart 1.

### CHART 1

CHART 1

| | ‘POULfeld’ | ‘POULlander’ |
|---|---|---|
| Flower bud color, | White Group 155C to | Red Group 46A. |
| as sepals divide to | Green White Group 155C | |
| 1/4 open. | | |
| Color, upper petal | White Group 155A. | Red Group 46B to 46C. |
| surface, upon | | |
| opening. | | |
| Petalage under | 40-50 petals. | Approx. 20 petals. |
| normal | | |
| conditions. | | |

Parents:

Seed parent.—An unnamed seedling.

Pollen parent.—An unnamed seedling.

Classification:

Botanical.—Rosa hybrida.

Commercial.—Floribunda.

### FLOWER AND FLOWER BUD

Blooming habit: Nearly Continuous.

Flower bud:

Size: Upon opening.—30 mm -35 mm in length from base of receptacle to end of bud.

Bud form.—Long, pointed ovoid bud with a high center.

Bud color.—As sepals unfold, White Group 155C. GreenWhite Group 155C at ¼opening. In cooler weather, intonations on guard petals of Green Group 143B and Greyed-Red Group 180B.

Sepals.—Yellow-Green Group 146B. With weak foliaceous appendages. Surfaces of sepals slightly pubescent. Limited numbers of stipitate glands present on margins.

Receptacle.—Surface: Generally smooth, at times glaucous. Shape: Urn-shaped Size: Medium, 7 mm (h)×6 mm (w). Color: Yellow-Green Group 144A 146C.

Peduncle.—Surface: Smooth. with some stipitate glands. Length: 30-40 mm average length. Color: Yellow-Green Group 146C, some with intonations of Greyed-Red Group 179A. Strength: Upright.

Borne.—Typically with 2-5 buds per flowering stem.

Flower bloom:

Fragrance.—Light floral and fruit fragrance.

Duration.—As a cut flower 6 to 8 days. The blooms have a duration on the plant of approximately 8 to 10 days. Petals fall cleanly away from plant.

Size.—Average flower diameter is 70-80 mm when open. p2 Form.—Shape of flower when viewed from the side: Upon opening, upper part: Convex. Upon opening, lower part: Convex Open flower, upper part: Flattened convex. Open flower, lower part: Flat to concave.

Petalage.—Very double. Average range: 40-50 petals under normal conditions with 2-6 petaloids.

Color:

Upon opening.—Outermost petals: Upper Surface: White Group 155A. Reverse Side: White Group 155A. Innermost petals.—Basal and mid-zone of petal with intonations of Yellow Group 2D. Upper Surface: White Group 155A Yellow Group 4C. Reverse Side: White Group 155A Yellow Group 4C.

Upon opening, basal petal spots.—Yellow intonation slightly stronger on innermost petals. Outermost petals: Outer Side: Yellow Group 1D. Inner Side: Yellow Group 1C. Innermost petals: Outer Side: Yellow Group 1C-1D. Inner Side: Yellow Group 1C-2C.

After opening, petals.—Upper Surface: White Group 155A. Reverse Side: White Group 155A.

After opening, basal petal spots.—Outer Side: Green-White Group 157D. Inner Side: Green-White Group 157D.

General Tonality: On open flower, White Group 155A. Tonality does not change.

Petals:

Petal Reflex.—Petals reflexed on outermost ⅓of petals, few petals double reflexed.

Petal Edge.—Uniform.

Shape.—Deltoid shaped.

Petaloids.—Present. Quantity 2-6.

Texture.—Above average thickness.

Arrangement.—Imbricated.

Reproductive Organs:

Pollen.—Color: Yellow-Orange Group 16C. Quantity: Average.

Anthers.—Size: Medium. Color, immature: Yellow-Orange Group 16C. on margin center, Yellow Group 1D. Color, mature: Greyed-Orange Group 164C. Quantity: Average to above average.

Filaments.—Color: Green-Yellow Group 1C.

Stigmas.—Slightly superior in location to anthers. Color: Yellow-Green Group 149D.

Styles.—Color: Green-White Group 157A. Other intonations: Limited intonations of Greyed-Red Group 179B immediately below stigma.

### PLANT

Plant growth: Vigorous. Plant is upright to bushy. When grown as a budded field grown plant on Rosa multiflora understock, after two growing seasons, the average height of the plant itself is 65 cm and the average width is 50 cm.

Stems:

Color.—Young wood: Yellow-Green Group 144B-146C. Older wood: Yellow-Green Group 146C.

Thorns.—Incidence: Few. Upper stems mostly thornless. Size: Average length: 7 mm -8 mm. Color: Older-Greyed-Orange Group 172C. Shape: Deeply concave.

Surface.—Young wood: Smooth. Older wood: Smooth.

Plant foliage: Normal number of leaflets on normal leaves in middle of the stem: 5 leaflets.

Leaf size.—Medium, 115-125 mm (1)×75-85 mm (w).

Abundance.—Average.

Color.—

Upper Leaf Surface.—Yellow-Green Group 147A.

Lower Leaf Surface.—Yellow-Green Group 146C.

Juvenile foliage.—Yellow-Green Group 144B.

Anthocyanin intonation.—Location: Leaflet, margins, petiole, rachis, stipules, stems, and underside of leaflet blade. Color: Greyed-Green Group 179A.

Plant leaves and leaflets:

Stipules.—Size: 18 mm -22 mm. Color: Yellow-Green Group 146B.

Presence of stipitate glands: On margins.

Petiole.—Length: 30 mm -35 mm. Color: Yellow-Green Group 146A. Underneath: Smooth, some petioles with 1 or 2 small prickles Margins: With stipitate glands.

Rachis.—Color: Yellow-Green Group 146A. Underneath: Smooth. With limited numbers of small prickles.

Margins.—With stipitate glands

Leaflet.—Edge: Finely serrated. Shape: Ovate to round. Texture: Leathery. Other: Moderately glossy.

Disease resistance: Above average resistance to mildew and rust under normal growing conditions in Jackson County, Oregon.

Winter hardiness: ‘POULfeld’ has shown to be winter hardy in Denmark and in Jackson County, Oregon.

1. A new and distinct variety of rose plant of the floribunda class, substantially as herein illustrated and described as a distinct and novel rose variety due to its abundant, white flowers, vigorous growth, disease resistance, and extended period of bloom:
